SEQUENCE LISTING (7 sequences)

SEQ ID NO: 1 - protein, 15 aa, Tolypocladium niveum
  MISC_FEATURE (1)..(1): ACETYLATION, pipecolic acid
  MOD_RES (2)..(2): Aib
  MISC_FEATURE (3)..(3): pipecolic acid
  MOD_RES (4)..(5): Aib
  MOD_RES (7)..(7): bAla
  MOD_RES (9)..(10): Aib
  MISC_FEATURE (11)..(11): pipecolic acid
  MOD_RES (12)..(12): Aib
  MOD_RES (15)..(15): BLOCKED, Aib
XAXAALAGAAXAGLA

SEQ ID NO: 2 - protein, 15 aa, Tolypocladium niveum
  MISC_FEATURE (1)..(1): ACETYLATION, pipecolic acid
  MOD_RES (2)..(2): Aib
  MISC_FEATURE (3)..(3): pipecolic acid
  MOD_RES (4)..(5): Aib
  MOD_RES (7)..(7): bAla
  MOD_RES (9)..(10): Aib
  MISC_FEATURE (11)..(11): pipecolic acid
  MOD_RES (12)..(12): Aib
  MOD_RES (15)..(15): BLOCKED, isovaline
XAXAALAGAAXAGLV

SEQ ID NO: 3 - protein, 15 aa, Tolypocladium niveum
  MISC_FEATURE (1)..(1): Acetylation, pipecolic acid
  MOD_RES (2)..(2): Aib
  MISC_FEATURE (3)..(3): pipecolic acic
  MOD_RES (4)..(4): isovaline
  MOD_RES (5)..(5): Aib
  MOD_RES (7)..(7): bAla
  MOD_RES (9)..(10): Aib
  MISC_FEATURE (11)..(11): pipecolic acid
  MOD_RES (12)..(12): Aib
  MOD_RES (15)..(15): BLOCKED, isovaline
XAXVALAGAAXAGLV

SEQ ID NO: 4 - protein, 15 aa, Tolypocladium niveum
  MISC_FEATURE (1)..(1): ACETYLATION, pipecolic acid
  MOD_RES (2)..(2): Aib
  MISC_FEATURE (3)..(3): pipecolic acid
  MOD_RES (4)..(5): Aib
  MOD_RES (7)..(7): bAla
  MOD_RES (9)..(10): Aib
  MISC_FEATURE (11)..(11): pipecolic acid
  MOD_RES (12)..(12): Aib
  MOD_RES (15)..(15): BLOCKED, isovaline
XAXAALAGAAXAALV

SEQ ID NO: 5 - protein, 15 aa, Tolypocladium niveum
  MISC_FEATURE (1)..(1): ACETYLATION, pipecolic acid
  MOD_RES (2)..(2): Aib
  MISC_FEATURE (3)..(3): pipecolic acid
  MOD_RES (4)..(4): isovaline
  MOD_RES (5)..(5): Aib
  MOD_RES (7)..(7): bAla
  MOD_RES (9)..(10): Aib
  MISC_FEATURE (11)..(11): pipecolic acid
  MOD_RES (12)..(12): Aib
  MOD_RES (15)..(15): BLOCKED, isovaline
  MOD_RES (15)..(15): BLOCKED, isovaline
XAXVALAGAAXAALV

SEQ ID NO: 6 - protein, 4 aa, Tolypocladium niveum
  MOD_RES (1)..(1): ACETYLATION, Aib
  MOD_RES (4)..(4): BLOCKED, isovaline
AGLV

SEQ ID NO: 7 - protein, 2 aa, Tolypocladium niveum
  MOD_RES (1)..(1): ACETYLATION, Leu
  MOD_RES (2)..(2): BLOCKED, isovaline
LV

(19) United States
(12) Patent Application Publication
(10) Pub. No.: US 2007/0249540 A1
(43) Pub. Date: Oct. 25, 2007

(54) Methods for Inhibiting Proteasome and Heat Shock Protein 90

(76) Inventor: Adonia Papathanassiu, Washington, DC (US)

Correspondence Address: Adonia Papathanassiu, PO Box 1001, Silver Spring, MD, 20910, US

(21) Appl. No.: 11/597,345

(22) PCT Filed: May 23, 2005
(86) PCT No.: PCT/US05/17913

§ 371 Date: Nov. 22, 2006

Related U.S. Application Data
(60) Provisional application No. 60/573,798, filed on May 24, 2004.

(51) Int. Cl. A61K 38/10; A61K 38/00; A61K 38/05; A61P 35/00; A61K 38/07

(52) U.S. Cl. 514/14; 514/18; 514/19; 514/2

(57) ABSTRACT

The present application provides methods for the inhibition of proteasome and heat shock protein Hsp90.

### CROSS-REFERENCE TO RELATED APPLICATIONS

The present patent application claims benefit of provisional patent application entitled “Methods for Inhibiting Proteasome and Heat Shock Protein 90” with filing date May 24, 2004 and patent application No. 60/573,798.

### BACKGROUND OF THE INVENTION

26S proteasome (also referred to simply as proteasome) is a multicatalytic protease responsible for the spatial and temporal destruction of proteins. This is a fundamental process used by the cell to dispose misfolded, damaged, or improperly assembled proteins and to modulate the levels of regulatory proteins that control basic cellular functions such as cell cycle progression, activation of transcription factors, and apoptosis. Proteasome is found in both the cytoplasm and the nucleus of all eukaryotic cells and is capable of rapid translocation between compartments in order to expedite cellular responsiveness to extracellular signals.

On the molecular level, proteasome is an extremely complex enzyme structured to prevent uncontrolled or inappropriate protein destruction. Its catalytic core, named 20S proteasome, resembles a hollow cylinder with the active sites sequestered in its interior cavity. This cylinder is flanked by two identical 19S regulatory proteins, which also control the access of substrates to the inner chamber of the core. In this system, destruction of selected proteins is secured by tagging these proteins with ubiquitin chains, a task accomplished by the ubiquitin enzymatic cascade. Once ubiquitinated, targeted proteins are readily recognized by receptors on the 19S complexes. The latter are also responsible for cleaving the ubiquitin chains away to be recycled back into the system, preparing the substrates for degradation by unfolding them, opening the channel that leads to the active sites of the core and catalyzing the translocation of denatured proteins in the inner chamber of the 20S proteasome. While the 20S proteasome is basically a multiprotease, the 19S regulatory proteins are ATPases, accomplishing the majority of their tasks in an ATP-dependent fashion.

Inhibition of proteasome has recently emerged as a major strategy for the development of anticancer therapies. This strategy is based on the observation that there is a marked variation in the levels of proteasomal activity between various cells types. For example, rapidly growing cells are more susceptible to proteasome inhibition than differentiated ones, while tumor cells exhibit increased proteolytic activity compared to their normal counterparts, an event not solely attributable to the uncontrolled proliferation of cancer cells. The genetic instability of tumor cells may require increased levels of protein degradation in order to remove misfolded and inappropriate proteins, whose accumulation is toxic to the cell. Certain types of cancer appear to be exquisitely sensitive to drugs that inhibit proteasome. These types include hematologic malignancies, colon cancer, head and neck squamous cell carcinoma and melanoma.

Bortezomib (VELCADE™, PS-341), a novel proteasome inhibitor, was recently approved for use in patients with refractory and relapsed multiple myeloma (Richardson et al Proteasome inhibition in hematologic malignancies, Ann Med 36(4):304-14 (2004)). Multiple myeloma is a form of leukemia arising from post-germinal mature B cells and characterized by the excess secretion of abnormal monoclonal immunoglobulins in the serum and urine. In multiple myeloma as well as in colon cancer, head and neck squamous cell carcinoma, and melanoma, the sensitivity of tumor cells to proteasome inhibitors is linked to prevention of NF-κB activation (Hochwald et al Antineoplastic therapy in colorectal cancer through proteasome inhibition, Am. Surg. 69(1):15-23 (2003); Amiri et al Augmenting chemosensitivity of malignant melanoma tumors via proteasome inhibition: implication for bortezomib (VELCADE, PS-341) as a therapeutic agent for malignant melanoma, Cancer Res. 64(14):4912-8 (2004)). NF-κB is constitutively activated in colorectal cancer, head and neck squamous cell carcinoma, and melanoma, where it contributes to the transcriptional activation of a variety of genes involved in proliferation, survival, and chemoresistance of the tumor cells (Lind et al Nuclear factor-kappa B is upregulated in colorectal cancer, Surgery 130(2):363-9 (2001); Chang and Van Waes, Nuclear factor-KappaB as a common target and activator of oncogenes in head and neck squamous cell carcinoma, Adv Otorhinolaryngol. 62:92-102 (2005); Amiri et al Augmenting chemosensitivity of malignant melanoma tumors via proteasome inhibition: implication for bortezomib (VELCADE, PS-341) as a therapeutic agent for malignant melanoma, Cancer Res. 64(14):4912-8 (2004)).

NF-κB exists in the cytoplasm as a complex with its inhibitor, IκB. Activation of NF-κB requires degradation of IκB, an event that allows NF-κB to translocate to the nucleus, where it initiates gene transcription. Degradation of IκB is mediated by the ubiquitin-proteasome system. Inhibitors of proteasome inhibit NF-κB activation by inhibiting IκB degradation and are expected to play a role in diseases characterized by constitutive activation of NF-κB. Such diseases include diabetes mellitus, renal failure, and inflammatory diseases such as atheroschlerosis, rheumatoid arthitis, and post-ischemic inflammation (Tas et al Signal transduction pathways and transcription factors as therapeutic targets in inflammatory disease: towards innovative antirheumatic therapy, Cur Phasm Des 11:581-611 (2005); Celec P., Nuclear factor kappa B-molecular biomedicine: the next generation, Biomedicine Pharmacotherapy 58:365-371 (2004); Zheng et al Post-ischemic inflammation: molecular mechanisms and therapeutic implications. Neurol Res. 26(8):884-92 (2004)).

The majority of proteasome inhibitors target the enzymatic activities of the core, mainly the ability to cleave after hydrophobic residues (chymotrypsin-like or CT-L), after basic residues (trypsin-like or T-L), or after acidic residues (peptidylglutamyl-peptide hydrolyzing (PGPH) or caspase-like). These activities have been assigned to distinct catalytic sites within the core.

Heat shock proteins (Hsps) are a family of housekeeping molecules that function as molecular chaperones to recognize proteins with abnormal conformations, prevent them from nonspecific aggregation, and support their conversion to a native, functional structure. Hsps are particularly vital to cells under conditions conducive to the production of abnormal proteins. They were first seen in cells exposed to elevated temperatures and they are known to be upregulated in cells responding to environmental stress. Because deleterious environmental conditions are commonly found in tumors, Hsps appear to play a significant role in cancer growth and progression, possibly by allowing cancer cells to successfully survive and adapt to a harmful milieu created by hypoxia, nutrient deprivation, accumulation of harmful metabolic by-products, and often exposure to chemotherapy and radiation.

One of the most prominent members of the growing number of proteins recognized as molecular chaperones is Hsp90. Hsp90 binds to proteins that are near native conformational state and promotes their appropriate structural folding, stable expression, and activity. The majority of the Hsp90 client proteins is involved in signal transduction; these proteins include serine-threonine kinases Raf-1 and Akt, the receptor tyrosine kinase Erb21Neu, mutated p53, and hormone receptors such as estrogen receptor (ER), androgen receptor (AR), and progesterone receptor (PR). Inhibition of Hsp90 leads to the destabilization of the client proteins followed by their ubiquitination and proteasomal degradation. Because so many of the Hsp90 substrate proteins are essential in promoting cell growth and survival of hormone-dependent cancers such as endometrial, ovarian, breast, prostate and lung cancer, these cancers are particularly sensitive to inhibition of Hsp90 (Neckers, L. Heat shock protein 90 is a rational molecular target in breast cancer, Breast Dis. 15:53-60 (2002); Solit et al. Hsp90 as a therapeutic target in prostate cancer, Semin Oncol 30(50:709-16 (2003); Stabile et al. Estrogen receptor pathways in lung cancer. Curr Oncol Rep. 6(4):259-67 (2004)). In these cancers, inhibition of Hsp90 results in inhibition of tumor cell proliferation (Gossett et al. 17-Allylamino-17-demethoxygeldanamycin and 17-NN-dimethyl ethylene diamine-geldanamycin have cytotoxic activity against multiple gynecologic cancer cell types, Gynecol Oncol 96(2):381-8 (2005)). When used in combination, inhibitors of Hsp90 are also known to increase the sensitivity of tumor cells to common cancer treatments including ionizing radiation, chemotherapy, and hyperthermia. For example, geldanamycin, a known Hsp90 inhibitor, sensitizes Bcr-Abl-expressing leukemia cells to taxol or doxorubicin treatment (Blagosklonny et al. The Hsp90 inhibitor geldanamycin selectively sensitizes Bcr-Abl-expressing leukemia cells to cytotoxic chemotherapy, Leukemia, 15(10):1537-43 (2001)). Inhibition of Hsp90 also synergizes with cisplatin (Bagatell et al. Hsp90 inhibitors deplete key anti-apoptotic proteins in pediatric solid tumor cells and demonstrate synergistic anticancer activity with cisplatin, Int J Cancer, 113(2):179-88 (2005)).

Due to the importance in the growth and survival of certain types of cancer, Hsps are often found upregulated in tumor cells. Hsp27 is a small molecular weight. Hsp, known to be involved in intracellular signaling and drug resistance (Fortin et al. Overexpression of the 27 KDa heat shock protein is associated with thermoresistance and chemoresistance but not with radioresistance, Int J Radiat Oncol Biol Phys, 46(5):1259-66 (2000)). It is found upregulated in lung carcinomas, osteosarcomas and in gynecological cancers such as breast and cervical cancer (Malusecka et al. Expression of heat shock proteins Hps70 and Hsp27 in primary non-small cell lung carcinomas. An immunohistochemical study, Anticancer Res. 21(2A):1015-21 (2001); Uozaki et al. Overexpression of resistance-related proteins (metallothioneins, glutathione-S-transferase pi, heat shock protein 27, and lung resistance-related protein) in osteosarcoma. Relationship with poor prognosis, Cancer, 79(12):2336-44; El-Ghobashy et al. Upregulation of heat shock protein 27 in metaplastic and neoplastic lesions of the endocervix, Int. J. Gynecol. Cancer, 15(3):503-9 (2005)). Upregulation of Hsp27 in human cancers often correlates with poor prognosis. Kindas-Mugge et al. reported that Hsp27 is an Hsp90 client protein (Kindas-Mugge et al. Characterization of proteins associated with heat shock protein hsp27 in the squamous cell carcinoma cell line A431. Cell Biol Int. 26(1):109-16 (2002)). Depletion of the intracellular levels of Hsp27 leads to reversal of resistance in multiple myelomas (Chauhan et al., 2-Methoxyestardiol and bortezomib/proteasome-inhibitor overcome dexamethasone-resistance in multiple myeloma cells by modulating Heat Shock Protein-27, Apoptosis, 9(2):149-55 (2004); Chauhan et al. Blockade of Hsp27 overcomes Bortezomib/proteasome inhibitor PS-341 resistance in lymphoma cells, Cancer Res, 63(19):6174-7 (2003)).

### BRIEF SUMMARY OF THE INVENTION

In accordance with the present invention, methods are provided for inhibiting proteasomal enzymatic activity, for inhibiting Hsp90 chaperone activity, and for treating human and animal cancers.

In an embodiment of the invention, methods are provided for inhibiting proteasomal enzymatic activity in a subject, such as a human or other animal, by administering to the subject one or more efrapeptin oligopeptides selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7. The efrapeptin oligopeptides may be administered in combination with a pharmaceutically acceptable carrier.

In a further embodiment of the invention, methods are provided for inhibiting tumor growth in a subject, such as a human or other animal, suffering from a cancer selected from group of cancers including but not limited to leukemia, colon cancer, head and neck squamous cell carcinoma, and melanoma by administering to the subject a composition containing one or more efrapeptin oligopeptides selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7. The efrapeptin oligopeptides may be administered in combination with a pharmaceutically acceptable carrier.

In another embodiment of the invention, methods are provided for inhibiting Hsp90 chaperone activity in a subject, such as a human or other animal, by administering to the subject one or more efrapeptin oligopeptides selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7. The efrapeptin oligopeptides may be administered in combination with a pharmaceutically acceptable carrier.

In yet another embodiment of the invention, methods are provided for inhibiting intracellular levels of Hsp27 in a subject, such as a human or other animal, by administering to the subject one or more efrapeptin oligopeptides selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7. The efrapeptin oligopeptides may be administered in combination with a pharmaceutically acceptable carrier.

In an additional embodiment of the invention, methods are provided for inhibiting tumor growth in a subject, such as a human or other animal, suffering from a cancer selected from group of cancers including but not limited to osteosarcoma, endometrial cancer, ovarian cancer, breast cancer, prostate cancer, and lung cancer by administering to the subject a composition containing one or more efrapeptin oligopeptides selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7. The efrapeptin oligopeptides may be administered in combination with a pharmaceutically acceptable carrier.

In a yet additional embodiment of the invention, methods are provided for enhancing the anti-tumor effect of an approved treatment, where the approved treatment is selected from a group of treatments that include ionizing radiation, hyperthermia, and chemotherapeutics taxol, doxorubicin, 5-fluorouracil, and cisplatin, given to a subject, such as a human or other animal, suffering from cancer, by administering to the subject a composition containing one or more efrapeptin oligopeptides selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7. The efrapeptin oligopeptides may be administered in combination with a pharmaceutically acceptable carrier. The efrapeptin oligopeptides may be administered before, concurrent with or after the administration of the approved treatment.

In a further embodiment of the invention, methods are provided for inhibiting NF-κB activity in a subject, such as a human or other animal, suffering from a disease, which disease is characterized by constitutive activation of NF-κB and is selected from a group of diseases including diabetes mellitus, renal failure, and inflammatory diseases such as atherosclerosis, rheumatoid arthritis, inflammatory bowel diseases and post-ischemic inflammation, by administering to the subject a composition containing one or more efrapeptin oligopeptides selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7. The efrapeptin oligopeptides may be administered in combination with a pharmaceutically acceptable carrier.

### BRIEF DESCRIPTION OF DRAWINGS

FIG. 1 is a list of efrapeptins corresponding to the amino acid sequences SEQ ID NO: 1, SEQ ID NO:2, SEQ ID NO: 3, SEQ ID NO: 4, SEQ ID NO: 5, SEQ ID NO: 6 and SEQ ID NO:7.

FIG. 2 is a graph depicting inhibition of chymotrypsin-like activity of purified 20S proteasome by SEQ ID NO: 2.

FIG. 3 is a graph depicting inhibition of chymotrypsin-like activity of 26S proteasome found in whole cell extracts of HT-29 colon cancer cells by SEQ ID NO: 2.

FIG. 4 is a graph depicting the levels of chymotrypsin-like activity in whole cell extracts of HT-29 colon cancer cells treated with SEQ ID NO: 2.

FIG. 5 is a Western blot depicting accumulation of polyubiquitinated proteins whole cell extracts of human umbilical vein endothelial cells (HUVECs) treated with SEQ ID NO: 2.

FIG. 6A is a Western blot showing the intracellular levels of IκB found in HUVECs stimulated with 4 ng/mL IL-1β in the absence and presence of pretreatment of HUVECs with SEQ ID NO: 2.

FIG. 6B is a Western blot showing the ubiquitination of IκB found in HUVECs stimulated with 4 ng/mL IL-1β in the absence and presence of pretreatment of HUVECs with SEQ ID NO: 2.

FIG. 7A is a Western blot showing the levels of Hsp90 complexed with mutant p53 in whole cell extracts of HT-29 cells treated with SEQ ID NO: 2.

FIG. 7B is a Western blot showing the intracellular levels of mutant p53 following overnight exposure of HT-29 colon cancer cells to various concentrations of SEQ ID NO: 2.

FIG. 8 is a Western blot showing the levels of Hsp27 complexed with Hsp90 in whole cell extracts of MCF-7 cells treated with SEQ ID NO: 2.

FIG. 9 is a graph depicting inhibition of tumor growth in C57BL/6 mice suffering from Lewis Lung Carcinoma by administering daily i.p. doses of 0.3 mg/Kg of SEQ ID NO: 2.

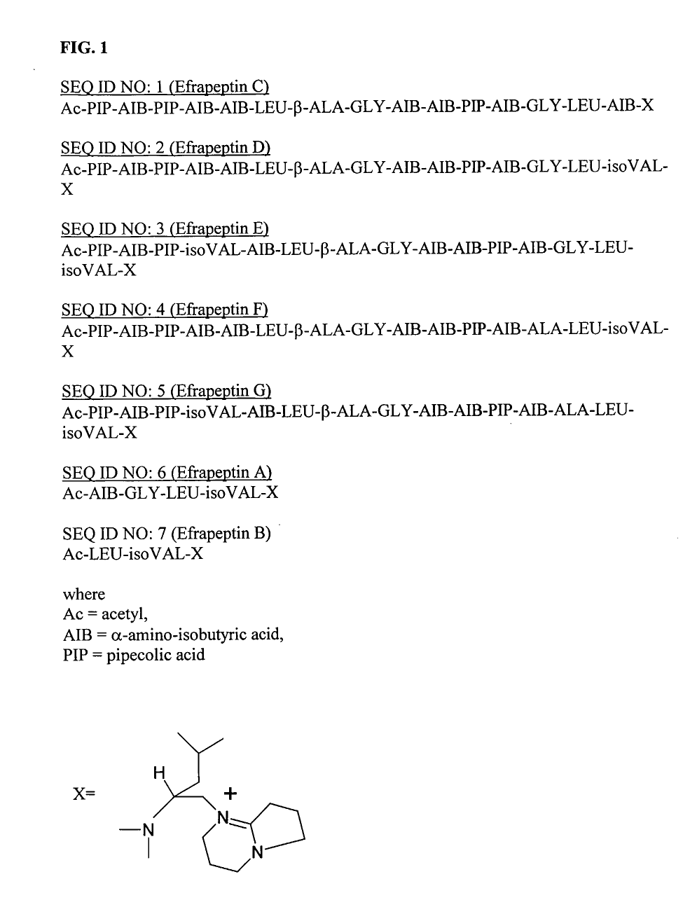

Compounds According to the Invention

Compounds, which are useful in accordance with the invention, are the efrapeptin oligopeptides shown herein as SEQ ID NO: 1, SEQ ID NO: 2, SEQ ID NO: 3, SEQ ID NO: 4, SEQ ID NO: 5, SEQ ID NO: 6, and SEQ ID NO:7.

### DETAILED DESCRIPTION OF THE INVENTION

Other objects, features and aspects of the present invention are disclosed in, or are obvious from, the following Detailed Description. It is to be understood by one of ordinary skill in the art that the present discussion is a description of exemplary embodiments only and is not intended as limiting the broader aspects of the present invention, which broader aspects are embodied in the exemplary construction.

The present invention comprises methods for inhibiting proteasomal enzymatic activities. It also comprises methods for inhibiting degradation of IκB. Furthermore it comprises methods for inhibiting Hsp90 chaperone activity and depleting the intracellular levels of Hsp90 client proteins. Finally, it comprises methods for inhibiting growth of human cancer cells. The treatment comprises the administration of an efrapeptin oligopeptide in sufficient amount to inhibit pathological conditions regulated by proteasome or Hsp90 activities. Each method of the present invention may be practiced in vitro, in vivo, ex vivo, and may further be practiced in a living subject such as human or other animal. The methods of the present invention may further be practiced on cells isolated from a subject in culture.

I. Definitions

The terms “a”, “an” and “the” as used herein are defined to mean one or more and include the plural unless the context is inappropriate.

The term “inhibitor” refers to a substrate that blocks or suppresses the activity of an enzyme and it includes reversible, irreversible, competitive, and noncompetitive inhibitors.

The term “inhibition” refers to the ability of an inhibitor to induce a decrease, reduction, suppression, depletion or downregulation of a cellular process including but not limited to an enzymatic activity such as Hsp90 chaperone activity, or a cellular event such as synthesis or degradation of a protein, or cellular proliferation.

The term “treatment” refers to the administration of a therapeutic modality with the purpose to prevent or delay disease manifestation, alleviate disease symptoms, halt the progression of a disease, or to cure.

The term “chymotrypsin-like” activity refers to the ability of proteasome to cleave after hydrophobic residues.

The term “peptides” relates to chains of amino acids whose alpha carbons are linked through peptide bonds formed by a condensation reaction between the carboxyl group of the alpha carbon of one amino acid and the amino group of the alpha carbon of another amino acid. A peptide has two terminal amino acids, one amino acid with a free amino-group called the amino- or N-terminus and one amino acid with a free carboxyl group called the carboxyl- or C-terminus. In a peptide, amino acids are numbered starting at the amino terminus and increasing in the direction of the carboxyl-terminus.

Peptides are produced chemically, recombinantly, or isolated from fungal cultures. Solid phase is the preferred method for chemical synthesis of peptides. It involves the attachment of the C-terminal amino acid to an insoluble support and the sequential addition of the remaining amino acids. An alternative method for synthesizing amino acids is the recombinant nucleic acid method, which involves the generation of a nucleic acid sequence encoding the peptide, followed by the expression of the peptide in a host and isolation and purification of the expressed peptide.

Efrapeptin oligopeptides described herein are isolated from cultures of entomopathogenic fungi including Tolycladium niveum. With the exception of efrapeptins A (SEQ ID NO: 6) and B (SEQ ID NO:7), efrapeptin oligopeptides are composed of 15 amino acids, which include common amino acids alanine, glycine, leucine and uncommon amino acids α-amino-isobutyric acid, β-alanine, and pipecolic acid. Their amino terminal is acetylated, while the carboxyl-terminal is blocked by N-peptido-1-isobutyl-2{1-pyrrole-(1,2-a)-pyrimidinium,2,3,4,5,6,7,8-hexahydro}-ethylamine (Kransoff et al., Antifungal and Insecticidal Properties of Efrapeptins: Metabolites of the Fungus Tolypocladium niveum, J. Invert. Path., 58, 180-188 (1991)). Efrapeptins C (SEQ ID NO: 1), D (SEQ ID NO: 2), E (SEQ ID NO: 3), F (SEQ ID NO: 4), and G (SEQ ID NO: 5) are nearly identical differing only in one amino acid. The difference rises either by the substitution of a isoValine by an 2-amino-isobutyric acid or by the substitution of an glycine by an alanine. In both cases, one apolar hydrophobic amino acid is substituted by another. Such substitutions are common in nature and they do not alter the overall functionality of a protein. For example, glutamate is often substituted by aspartate, both negatively charged amino acids. Consequently, efrapeptins C (SEQ ID NO: 1), E (SEQ ID NO: 3), F (SEQ ID NO: 4), and G (SEQ ID NO: 5) are expected to have properties similar to those of efrapeptin D (SEQ ID NO: 2). Efrapeptin A (SEQ ID NO: 6) and Efrapeptin B (SEQ ID NO:7) are 100% homologous with sequences of Efrapeptin D (SEQ ID NO: 2). Peptides with sequences homologous to those efrapeptin oligopeptides described herein are also included in the present invention. Homologues are peptides with amino acid sequences that have sequence identity or homology with amino acid sequence of efrapeptin oligopeptides. “Sequence identity” and “sequence homology” as applied to an amino acid sequence herein is defined as a sequence with at least about 90%, 91%, 92%, 93%, or 94% sequence identity, and more preferably at least about 95%, 96%, 97%, 98%, or 99% sequence identity to another amino acid sequence, as determined, for example, by the FASTA search method in accordance with Pearson and Lipman, Proc. Natl. Acad. Sci. USA 85, 2444-2448 (1988). Pharmaceutical compositions in accordance with the present invention are administered at suitable doses and dosage regimes, which are determined by conventional dose-escalating techniques known to those of ordinary skill in the art. Generally, the treatment is initiated at less than the optimum dose and is incrementally increased until the optimum effect is achieved. The optimum dose for any given subject will depend on a number of factors including age, body weight, gender, diet, time and route of administration, the rate of excretion of the drug, and the severity of the disease. The present methods involve the administration of 0.05 mg to 100 mg per kilogram body weight per day of the compounds described herein. Preferably, the dosage levels are from 0.5 mg to 10 mg per kilogram of body weight per day and, more preferably, are from 1 mg to 5 mg of body weight per day. In addition, the compounds of the present invention may be administered at semi-weekly, weekly, semi-monthly, or monthly levels.

II. Suitable Methods for Practicing the Invention

Inhibition of 20S Proteasomal Enzymatic Activities

The enzymatic activities of 20S proteasome are determined via hydrolysis of fluorogenic substrates specific for each activity of the enzyme. Typically, fluorogenic peptides Suc-LLVY-AMC (Boston Biochem, Cambridge, Mass., Cat. No: S-280) Z-LLE-AMC (Boston Biochem, Cat. No. S230) and Boc-LSTN-AMC (Sigma Aldrich, St. Louis, Mo., Cat. No. B4636) are employed for measuring the chymotrypsin-like, caspase-like, and trypsin-like activities of proteasome, respectively. Production of released AMC (amido-coumarin) groups in an assay buffer (50 mM tris-HCl pH 7.5) containing 0.04% SDS is estimated using a fluorometer with an excitation filter of 380 nm and an emission filter of 460 μm. Presence of 0.04% SDS is required because uncomplexed 20S protease exists in a latent form and requiring chemical activation.

Inhibition of 26S Proteasomal Enzymatic Activities

The enzymatic activities of 26S proteasome are determined as previously described for 20S without chemical activation with SDS.

Accumulation of Ubiquitinated Proteins.

Inhibition of proteasomal activity prevents degradation of proteins and leads to accumulation of ubiquitinated proteins. To detect accumulation of such proteins, cells are grown in 100 mm dishes to 60% confluency. Subsequently, they are treated with various concentrations of a proteasomal inhibitor for 30 min and lysed in a suitable buffer. Proteins from whole cell extracts are then analyzed with SDS-PAGE and visualized by Western blotting using an appropriate anti-ubiquitin antibody.

Inhibition of NF-κB Activation.

Endothelial cells play a key role in mediating the inflammatory response. In the presence of pro-inflammatory cytokines such as IL-1 or TNF-α, endothelial cells degrade IκB, which leads to the nuclear translocation of NF-κB, and transcription of such inflammatory proteins as cell adhesion molecules and chemotactic cytokines [Wrighton et al. Inhibition of endothelial cell activation by adenovirus-mediated expression of I kappa B alpha, an inhibitor of the transcription factor NF-kappa B. J Exp Med. 183(3):1013-22 (1996)]. Inhibition of IκB degradation in endothelial cells activated with a pro-inflammatory cytokine suppresses the nuclear translocation of NF-κB and retards the inflammatory response. Inhibition of NF-κB activation can be determined by monitoring with Western immunoblotting the degradation of IκB after treatment of endothelial cells with a pro-inflammatory cytokine.

Inhibition of Hsp90.

Inhibition of Hsp90 chaperone activity is measured by determining the amount of Hsp90 in complex with a client protein such as mutant p53 after treatment with an Hsp90 inhibitor. Specifically, cells routinely cultured to 60% confluency are briefly exposed to various concentrations of an Hsp90 inhibitor. The cells are then lysed and the Hsp90 client protein is immunoprecipitated from equal amounts of whole cell extracts using a suitable antibody bound to agarose beads. Hsp90 complexed to the client protein is then eluted in a SDS sample buffer, analyzed with SDS-PAGE and visualized by Western blotting.

Inhibition of Hsp90 is also inferred through degradation of a client protein. Briefly, cells are treated with various concentrations of an Hsp90 inhibitor for at least 24 hrs. Subsequently, the cells are lysed using a suitable lysis buffer and equal amounts of whole cells extracts of treated cells are subjected to SDS-PAGE. The levels of the client protein in the cell lysates are determined by Western blotting.

Inhibition of Tumor Cell Proliferation

Tumor cells are allowed to proliferate in the presence of an inhibitory agent for 48 to 96 hrs. At the end of the assay, tumor cell proliferation in the presence of the inhibitory agent is determined either by cell counting or BrdU incorporation and expressed as % of Control Proliferation, where the term “Control Proliferation” refers to tumor cell proliferation in the absence of any inhibitory or other agents.

Inhibition of Tumor Growth

Tumor cells are injected s.c. onto the appropriate mouse background and allowed to proliferate until tumors become palpable (typical size 50-100 mm³). Treatment is then initiated by administering to a group of mice either an appropriately formulated active agent at a predetermined dosage regime or diluent control. Tumor volumes are assessed according to the formula:
Tumor volume=length×width²×π/6

The experiment is terminated when the control mice reach a certain tumor volume.

Administration

The compositions described previously may be administered by the topical, oral, rectal or parenteral (intravenous, subcutaneous or intramuscular) route. They may also be incorporated into biodegradable polymers for sustained release and implanted at the disease site. The dosage of the compositions depends on the condition treated, the activity of the drug used, the route of administration, and other clinical factors such as severity of the disease and weight of the patient. The compositions may administer as a bolus injection, or every day or intermittently every other day, every week, semi-monthly, monthly, etc. The compositions are formulated in ways suitable for the specific route of administration. Formulations suitable for oral administration include capsules, cachets or tablets containing a predetermined amount of the active ingredient, powder or granules, solutions, suspensions, and emulsions in a mixture with an organic or inorganic carrier or excipient. Such carriers comprise solvents, dispersion media, vehicles such as liposomes and nanocapsules, coatings, diluents, colloids and other. Formulations suitable for topical administration in the mouth include lozenges, pastilles, and mouthwashes. Formulations suitable for topical administration to the skin include ointments, creams, gels, pastes, and transdermal patches. Formulations for rectal administration may be presented as a suppository with a suitable base, while vaginal administrations maybe presented as pessaries, tampons, creams, gels, pastes, foams, and sprays comprising the active ingredient in an appropriate carrier. Formulations suitable for parenteral administration include aqueous and non-aqueous sterile injection solutions presented in unit-dose or multi-dose containers. It should be also understood that, in addition to the ingredients mentioned above, formulations of this invention might include other agents conventional in the art having regard to the type of formulation in question.

The invention is further understood by the following non-limiting examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

Inhibition of Chymotrypsin-Like Activity of 20S Proteasome by SEQ ID NO 2.

0.1 μg of purified rabbit 20S proteasome (from Boston Biochem) were incubated for 30 min at 37° C. with 200 μM Suc-LLVY-AMC (from Sigma Aldrich) in the absence and presence of various concentrations of SEQ ID NO: 2 in 100 μL of assay buffer (50 mM Tris-HCl pH7.5, 0.04% SDS). After incubation, production of hydrolyzed AMC groups was measured using a multi-well CytoFluor™2300 Fluorescence Measurement System (Millipore) with an excitation filter of 380 nm and an emission filter of 460 nm. Reactions were performed in triplicates. Experiments were repeated multiple times. Data are presented as % of control fluorescence (absence of SEQ ID NO: 2) and shown as MEAN±SD.

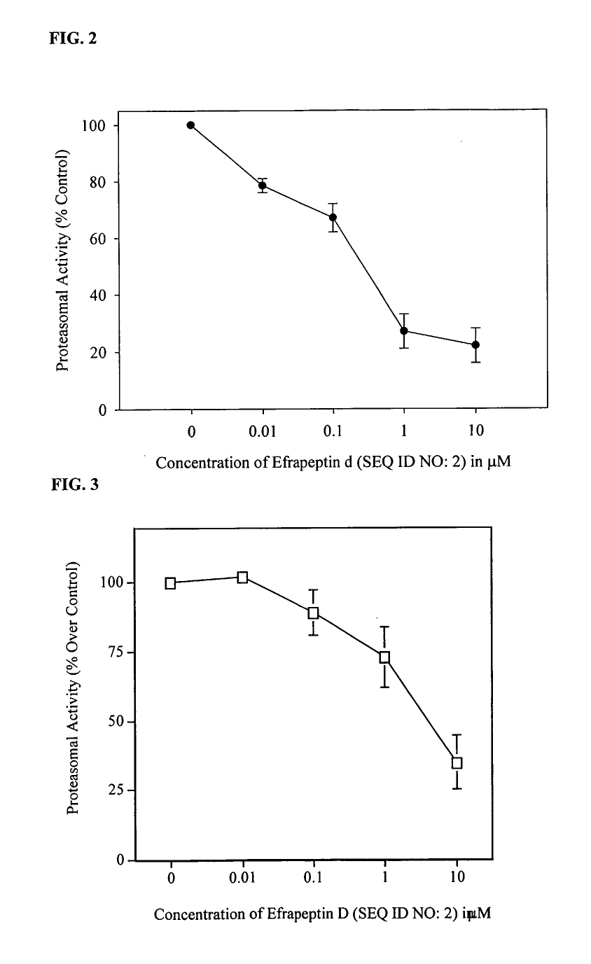

SED ID NO: 2 inhibited chymotrypsin-like activity of purified 20S proteasome in a concentration dependent manner (FIG. 2).

### EXAMPLE 2

Inhibition of Chymotrypsin-Like Activity of 26S Proteasome Present in HT-29 Whole Cell Extracts by SEQ ID NO 2.

25 μg of whole cell extracts obtained from untreated HT-29 cells were incubated for 30 min at 37° C. with 200 μM Suc-LLVY-AMC in the absence and presence of various concentrations of SEQ ID NO: 2 in 100 μL of assay buffer (50 mM Tris-HCl pH 7.5). After incubation, inhibition of chymotrypsin-like activity was determined and expressed as previously described.

SED ID NO: 2 inhibited chymotrypsin-like activity of 26S proteasome present in whole cell extracts obtained from untreated HT-29 cells in a concentration dependent manner (FIG. 3).

### EXAMPLE 3

Chymotrypsin-Like Activity of 26S Proteasome Present in Whole Cell Extracts Obtained from HT-29 Cells Treated with SEQ ID NO: 2.

HT-29 cells were treated with various concentrations of SEQ ID NO: 2 for 24 hrs. The cells were then washed 2× with ice-cold PBS and lysed with sonication (3 pulses, 5 sec/pulse) in 10 mM Tris-HCl pH 7.5 containing 2 mM ATP. BCA assay was then employed to determine the proteins levels in the whole cell extracts. Subsequently, 25 μg of whole cell extracts from each treatment group were incubated for 30 min at 37° C. with 200 μM Suc-LLVY-AMC in 100 μL of assay buffer (50 mM Tris-HCl pH 7.5). After incubation, the levels of chymotrypsin-like activity in each sample were determined and expressed as previously described.

HT-29 cells treated with SEQ ID NO: 2 for 24 hrs possessed reduced levels of chymotrypsin-like proteasomal activity (FIG. 4).

### EXAMPLE 4

Accumulation of Ubiquitinated Proteins in HUVECs Treated with SEQ ID NO: 2.

HUVECs were grown in 100 mm² dishes, treated for 30 min with various concentrations of SEQ ID NO: 2, and lysed at a rate of 12,500 cells per μl of lysis buffer. Whole cell extracts corresponding to 60,000 cells were loaded per lane, analyzed by SDS-PAGE, and visualized using a rabbit anti-ubiquitin ab.

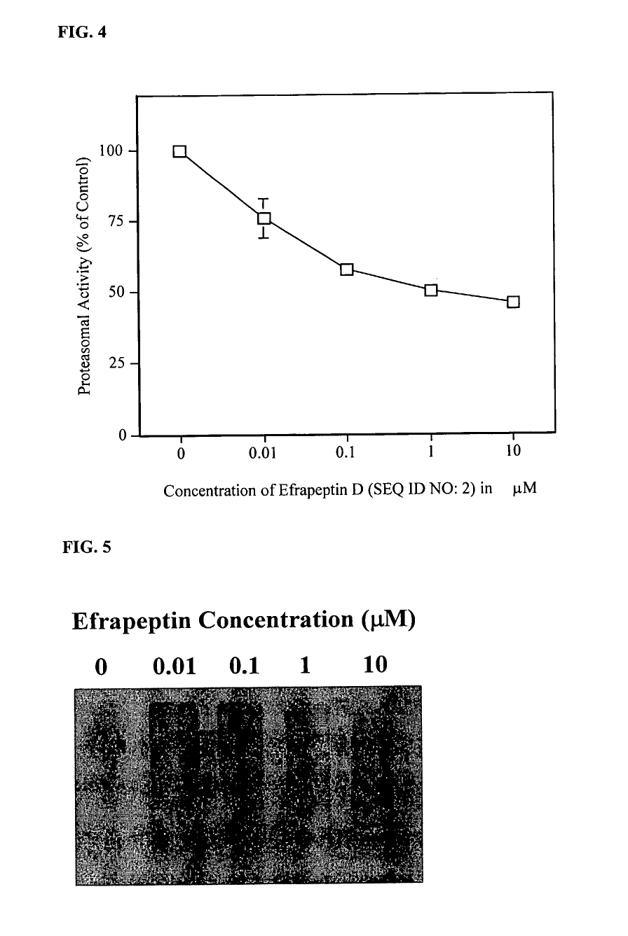

FIG. 5 indicates accumulation of ubiquitinated proteins in HUVECs after treatment with SEQ ID NO: 2.

### EXAMPLE 5

Accumulation of IκB in IL-1β-Activated Endothelial Cells Treated with SEQ ID NO: 2

Human umbilical vein endothelial cells (HUVECs) were cultured in 100 mm², pretreated with 10 μM of SEQ ID NO: 2 for 45 min, and treated with 4 ng/mL IL-1β for 30 min. Subsequently, the cells were lysed as previously described. After immunoprecipitation of IκB protein from equal amounts of cell lysates of the IL-1β-activated HUVECS, the protein was subjected to SDS-electrophoresis and Western immunoblotting.

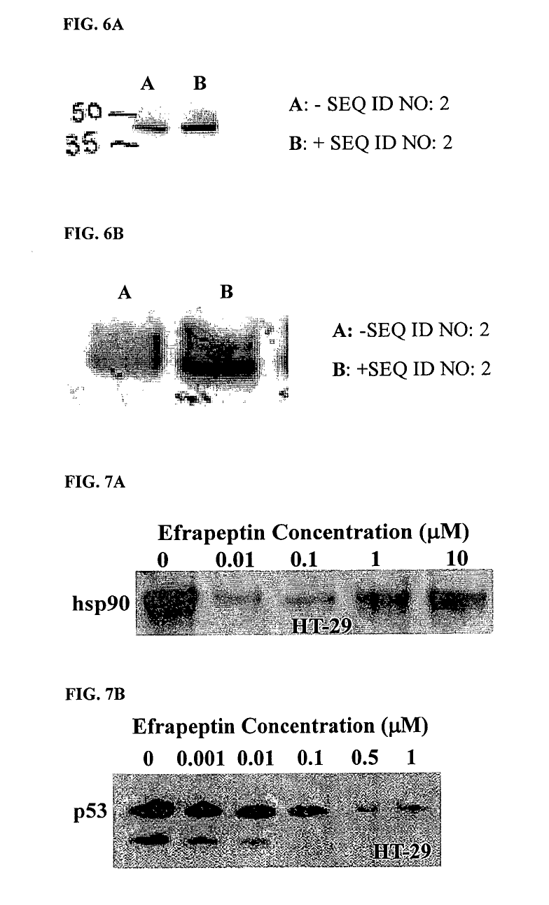

FIG. 6A shows accumulation of IκB, when IL-1β-activated HUVECs were pretreated with SEQ ID NO: 2.

To determine whether the accumulation of IκB was linked to inhibition of proteasome by SEQ ID NO: 2, the experiment was repeated. Ubiquitinated proteins present in equal amounts of cell lysates of the IL-1-activated HUVECS were immunoprecipitated and then subjected to SDS-electrophoresis and Western immunoblotting using an anti-IκB monoclonal antibody.

FIG. 6B shows ubiquitination of IκB, when IL-1β-activated HUVECs were pretreated with SEQ ID NO: 2.

### EXAMPLE 6

Levels of Hsp90 Complexed with Mutant p53 after Treatment of HT-29 Cells with SEQ ID NO 2.

HT-29 cells were treated with various concentrations of SEQ ID NO: 2 for 10 min and lysed as previous described. Mutant p53 was immunoprecipitated from 650 μg of whole cell extract using a mouse anti-p53 ab (1 μg/sample) in the presence of 10 mM molybdate. Immunoprecipitated p53 was then subjected to SDS-PAGE electrophoresis and Western blot analysis for complexing with Hsp90 using a rabbit anti-Hsp90 ab.

FIG. 7A indicates inhibition of Hsp90 complexing with mutant p53 after transient exposure of HT-29 cells to SEQ ID NO: 2.

### EXAMPLE 7

Levels of Mutant p53 after Treatment of HT-29 Cells with SEQ ID NO: 2.

HT-29 cells were treated with various concentrations of SEQ ID NO: 2 for 24 hrs. and lysed as previous described. Equal amounts of whole cells extracts were analyzed by SDS-PAGE and the levels of mutant p53 in the cells lysates were visualized by Western blotting.

FIG. 7B indicates degradation of mutant p53 following treatment of HT-29 cells with SEQ ID NO: 2.

### EXAMPLE 8

Levels of Hsp90 Complexed with Hsp27 after Treatment of HT-29 Cells with SEQ ID NO: 2.

MCF-7 cells were treated with various concentrations of SEQ ID NO: 2 for 10 min and lysed as previous described. Hsp90 complexes were immunoprecipitated from 2 mg of whole cell extract using a rabbit anti-Hsp90 ab (1 μg/sample) in the presence of 10 mM molybdate. Immunoprecipitated Hsp90 was then subjected to SDS-PAGE electrophoresis and Western blot analysis for complexing with Hsp27 using a mouse anti-Hsp27 ab.

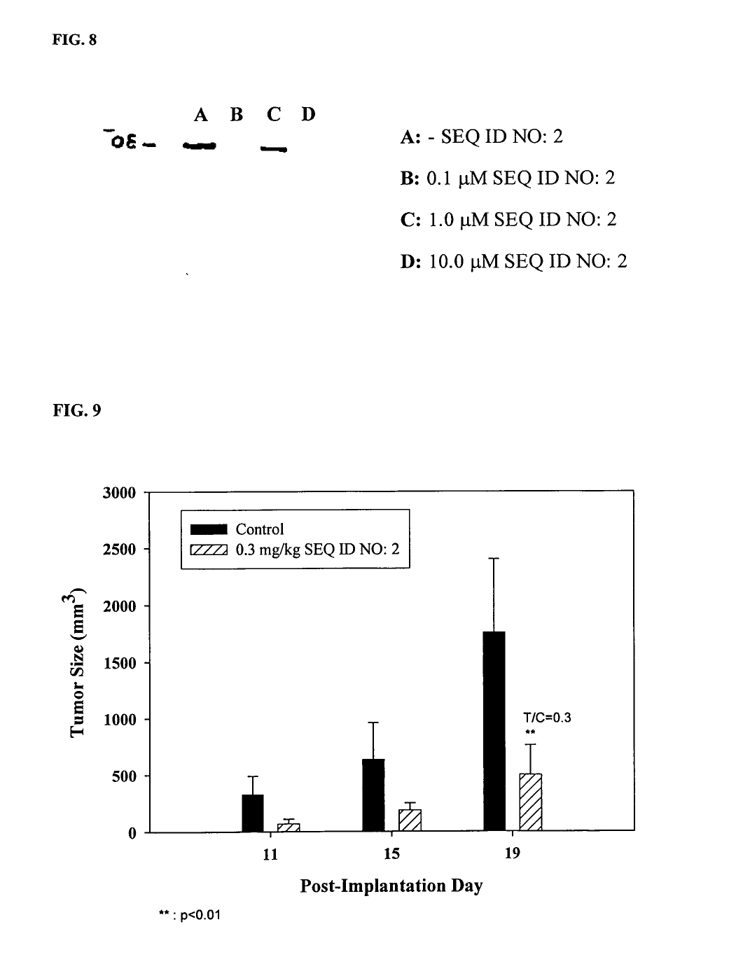

FIG. 8 indicates inhibition of Hsp90 complexing with Hsp27 after transient exposure of MCF-7 cells to SEQ ID NO: 2

### EXAMPLE 9

Inhibition of Tumor Cell Proliferation

SK-MES-1 lung cancer, HT-29 colon cancer and MCF-7, MDA-MB-231, MDA-MB-453 breast cancer cells were plated onto 24 well-plates at a cell density of 125,000 cells per well and allowed to adhere overnight. Subsequently, the tumor cells were treated with various concentrations of SEQ ID NO: 2 for 48 to 72 hrs. At the end of the treatment, cell proliferation was determined by cell counting.

SEQ ID NO: 2 suppressed proliferation of the cancer cell lines with the IC₅₀ values reported in Table I. IC₅₀ value is defined as the concentration of SEQ ID NO: 2 required to inhibit 50% of tumor cell proliferation.

TABLE I
Inhibition of Tumor Cell Proliferation by SEQ ID NO: 2

| Cell Type | Cell Name | IC₅₀ (μM) |
|---|---|---|
| Colon Cancer | HT-29 | 0.01-0.05 |
| Breast Cancer | MCF-7 | 0.05 |
| | MDA-MB-231 | 5 |
| | MDA-MD-453 | <0.001 |
| Lung Cancer | SK-MES-1 | 5 |
| Ovarian Cancer | SK-OV-3 | 5 |

### EXAMPLE 10

Sensitization of MCF—Breast Cancer Cells to the Cytotoxic Effects of Taxol.

MCF-7 breast cancer cells were plated onto a 24-well plate at a density of 208,000 cells per well and allowed to adhere overnight. Subsequently, the cells were treated with 0.01 μM taxol, a concentration known to inhibit 50% of MCF-7 cell proliferation. 6 hrs after the addition of taxol, half of the wells received 0.001 μM SEQ ID NO: 2. At a concentration of 0.001 μM, SEQ ID NO: 2 is known to have no inhibitory effects on MCF-7 cell proliferation. Cell proliferation of control (untreated) MCF-7 cells and MCF-7 cells treated either with Taxol or SEQ ID NO: 2 alone or with the combination of Taxol and SEQ ID NO: 2 was determined.

Table II shows sensitization of MCF-7 breast cancer cells to the cytotoxic effect of Taxol in the presence of a concentration of SEQ ID NO: 2 that has no effect on MCF-7 cell proliferation.

TABLE II
MCF-7 Proliferation in the Presence of Taxol and SEQ ID NO: 2

| Treatment | % Control Proliferation |
|---|---|
| 0.001 μM SEQ ID NO: 2 | 100 |
| 0.01 μM Taxol | 52 |
| 0.001 μM SEQ ID NO: 2 + 0.01 μM Taxol | 36 |

### EXAMPLE 111

Inhibition of Tumor Growth in Mice Bearing Lewis Lung Carcinoma Primary Tumors

A suspension of 1×10⁶ Lewis Lung carcinoma cells were injected s.c. into C57BL/6 mice (two groups; n=9). Treatment was initiated 7 days later. Mice received daily i.p. doses of 0.3 mg/kg of SEQ ID NO: 2 or diluent control for 12 days.

FIG. 9 shows tumor measurements on days 11, 15, and 19 post-implantation. The average tumor volume of control mice was 1760 mm³, while the average tumor volume of mice treated with 0.3 mg/kg SEQ ID NO: 2 was 500 mm³, a 70% reduction (p<0.01, Student's t-test); T/C=0.3, where T is the average tumor size of treated mice and C is the average tumor size of control mice.

All publications and patents referred to herein are expressly incorporated by reference in their entirety.

I claim:

1. A method for inhibiting proteasome in a subject, comprising administering to a subject in need thereof a therapeutically effective amount of an efrapeptin oligopeptide, thereby inhibiting proteasome in a subject.

2. The method of claim 1, wherein said subject is a human.

3. The method of claim 1, wherein said therapeutically effective amount is between about 0.1 mg/kg and about 10 mg/kg of body weight.

4. The method of claim 1, wherein the efrapeptin oligopeptide is selected from the group consisting of SEQ ID NO: 1, SEQ ID NO: 2, SEQ ID NO: 3, SEQ ID NO: 4, SEQ ID NO: 5, SEQ ID NO: 6 and SEQ ID NO: 7.

5. The method of claim 1, wherein said subject has a cancer selected from the group consisting of leukemia, colon cancer, head and neck squamous cell carcinoma and melanoma.

6. The method of claim 1, wherein the efrapeptin oligopeptide is administered in combination with a second therapy, wherein said second therapy is selected from the group consisting of ionizing radiation, doxorubicin, 5-fluorouracil and cisplatin, and wherein said second therapy is administered before, concurrent with or after administration of said efrapeptin oligopeptide.

7. The method of claim 1, wherein the subject has constitutive activation of NF-κB.

8. The method of claim 1, wherein the subject has diabetes mellitus, renal failure, or an inflammatory disease selected from the group consisting of atherosclerosis, rheumatoid arthritis, inflammatory bowel diseases and post-ischemic inflammation.

9. A method for inhibiting Hsp90 in a subject, comprising administering to a subject in need thereof a therapeutically effective amount of an efrapeptin oligopeptide, thereby inhibiting Hsp90 in a subject.

10. The method of claim 9, wherein said subject is a human.

11. The method of claim 9, wherein said therapeutically effective amount is between about 0.1 mg/kg and about 10 mg/kg body weight.

12. The method of claim 9, wherein the efrapeptin oligopeptide is selected from the group consisting of SEQ ID NO: 1, SEQ ID NO: 2, SEQ ID NO: 3, SEQ ID NO: 4, SEQ ID NO: 5, SEQ ID NO: 6 and SEQ ID NO: 7.

13. The method of claim 9, wherein the subject has a cancer selected from the group consisting of osteosarcoma, endometrial cancer, ovarian cancer, breast cancer, prostate cancer, and lung cancer.

14. The method of claim 9, wherein the efrapeptin oligopeptide is administered in combination with second therapy, wherein said second therapy is selected from the group consisting of ionizing radiation, hyperthermia, and chemotherapeutics taxol, doxorubicin, 5-fluorouracil, and cisplatin, and wherein said second therapy is administered before, concurrent with or after administration of said efrapeptin oligopeptide.

15. A method for inhibiting proteasome and Hsp90 in a subject, comprising administering to a subject in need thereof a therapeutically effective amount of an efrapeptin oligopeptide, thereby inhibiting proteasome and Hsp90 in a subject.

16. The method of claim 15, wherein said subject is a human.

17. The method of claim 15, wherein said therapeutically effective amount is between about 0.1 mg/kg and about 10 mg/kg body weight.

18. The method of claim 15, wherein the efrapeptin oligopeptide is selected from the group consisting of SEQ ID NO: 1, SEQ ID NO: 2, SEQ ID NO: 3, SEQ ID NO: 4, SEQ ID NO: 5, SEQ ID NO: 6 and SEQ ID NO: 7.
